(19) United States
(12) Patent Application Publication
(10) Pub. No.: US 2005/0244960 A1
(43) Pub. Date: Nov. 3, 2005

(54) Preparation of type II pneumocytes from stem cells

(75) Inventors: Anne Elizabeth Bishop, London (GB); Julia Margaret Polak, London (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NOVATHERA LIMITED, LONDON (GB)

(21) Appl. No.: 10/523,858

(22) PCT Filed: Aug. 6, 2003
(86) PCT No.: PCT/GB03/03407

§ 371 Date: Apr. 1, 2005

(30) Foreign Application Priority Data
Aug. 7, 2002 (GB) 0218332.5

(51) Int. Cl. C12N 5/08; A61K 45/00

(52) U.S. Cl. 435/366; 424/93.7

(57) ABSTRACT

Stem cells are induced in culture to form cells which express surfactant protein C (SPC), thereby offering a convenient and abundant supply of alveolar tissue without requiring lung donation or the use of cadavers. Stem cells are cultured to give embryoid bodies, which are they maintained under conditions which cause them to differentiate into cells which express SPC. SAGM medium has been found to be particularly effective for causing differentiation of the embryoid bodies.

### TECHNICAL FIELD

This invention is in the field of lung biology, and more particularly alveolar epithelial cells.

### BACKGROUND ART

The epithelium of the distal lung is highly specialised and is composed mainly of type I and type II pneumocytes. Type I alveolar epithelial (AE1) cells are particularly susceptible to damage and, following their loss during conditions of peripheral lung injury or disease, type II cells undergo a compensatory mechanism of proliferation and differentiation to a type I phenotype [1]. The alveolar type II (AE2) cell is also responsible for the synthesis and secretion of pulmonary surfactant, a complex mixture of phospholipids and proteins (e.g. surfactant protein C [2,3,4]) known to be critical for reducing surface tension at the air-liquid interface. Thus, type II cells are crucial to the natural regenerative process of the peripheral, gas-exchange component of the lung, and they have been described as defenders of the alveolus [5].

Because of their importance, the purification of AE2 cells has previously been the topic of active research. Reference 6 describes techniques for isolating and purifying AE2 cells from lung tissue. The purified cells are said to be useful for bioassay systems to monitor the effects of occupational or environmental pollutants on alveolar pneumocytes, and for studying the etiology of pulmonary disease in the alveolar region of the lung.

Other methods for purifying AE2 cells from lungs are described in references 7 to 10. Reference 9 suggests the transplantation of cells capable of regenerating lung alveolar surface for stimulating the growth of lung alveolar surface in patients.

AE2 cells thus have great potential both for therapeutic use and for investigating lung biology. Human cells are, however, in short supply as they can be prepared only from donated lung tissue. Lung tissue which is surgically removed is normally diseased in AE2 cells and thus cadavers are the only realistic supply. It is an object of the invention to provide efficient methods for producing AE2 cells which do not require lung tissue donation or the use of cadavers.

### DISCLOSURE OF THE INVENTION

The invention is based on the finding that embryonic stem cells can be induced in culture to form cells which express surfactant protein C (SPC), which is a characteristic marker for AE2 cells [11], thereby offering a convenient and abundant supply of material for study and transplant.

Thus the invention provides a process for causing a stem cell to differentiate into a cell which expresses surfactant protein C, the process comprising the steps of: (a) culturing the stem cell to give an embryoid body; and (b) culturing the embryoid body under conditions which cause it to differentiate into cells which express surfactant protein C. SPC^{+ve} cells formed in this way can then be recovered from the culture medium.

Differentiation Methods

Step (a) of the process of the invention involves the formation of embryoid bodies (EBs). These are aggregates of cells which are formed when embryonic stem (ES) cells, embryonic germ (EG) cells, or embryonal carcinoma (EC) cells are grown in suspension culture (e.g. when plated on a non-adhesive surface). They are widely recognised in the art and can be produced routinely [e.g. refs. 12 to 15] from both human [e.g. refs. 16 to 21] and mouse cells.

In step (a), stem cells will typically be grown initially in suspension culture (e.g. in a vessel which does not favour cell attachment, such as a petri dish), but during step (a) EBs will generally become non-dispersed and adherent. Techniques such as microbead culture [15] may also be used. A typical starting concentration is around 5×10⁴ cells per 10 ml of culture medium.

If starting stem cells are in adherent culture, they must be disengaged prior to step (a). Standard methods involving the use of enzymatic treatment (e.g. with trypsin, papain, collagenase etc.), mechanical disaggregation and/or metal ion chelators (e.g. EDTA, EGTA etc.) etc. can be used to disengage the cells.

Taking time zero as the time at which culture conditions are changed to favour EB formation (e.g. removal of LIF for murine ES cells), step (a) will generally last between 15 and 60 days, more typically at least 25 days (e.g. at least around 28-35 days). Longer periods of culture favour the development of endoderm, from which lung epithelium derives.

Step (a) may be performed in the presence or absence of serum. If serum is not used, a serum replacement may be used instead. The absence of serum avoids the introduction of undefined or random factors which may promote the formation of non-AE2 cell types. Its absence is not essential, however, and a typical medium for use in step (a) is G-MEM supplemented with serum, glutamine, 2-mercaptoethanol and antibiotics. Fresh medium may be supplied during step (a).

In step (b), EBs differentiate into SPC^{+ve} cells. SPC^{+ve} cells may develop with low efficiency in culture using the same medium as used in step (a) i.e. when steps (a) and (b) are separate phases of an essentially single continuing culture process. A low inherent differentiation efficiency may be acceptable when other techniques (e.g. lineage selection or cell enrichment as described below) are used, but it is preferred to increase differentiation efficiency by subjecting the EBs from step (a) to suitable differentiation conditions e.g. exposure to suitable differentiation factors. Such factors will typically be added to the medium in which the EBs are already being cultured or maintained. Alternatively, the EBs may be transferred into a new medium for exposure to the factors, and the new medium may be based on the same or different basal medium as used in step (a). If adherent EBs are to be transferred, they will first be disengaged as described above.

Exposure to ‘SAGM’ medium has been found to be particularly effective for causing differentiation in step (b). SAGM medium is part of the Small Airway Epithelial Cell Growth Media System supplied by Clonetics [22] for the culture of small airway epithelial cells (SAECs). SAGM consists of a serum-free basal medium (‘SABM’) supplemented with: BPE (bovine pituitary extract), hydrocortisone, HEGF (human recombinant epidermal growth factor), epinephrine, transferrin, insulin, retinoic acid, triiodothyronine, gentamicin, amphotericin-B and BSA-FAF (bovine serum albumin-fatty acid free). The medium generally has a pH in the range 7.4-7.6 and an osmolality of 255-265 mOsm/kg.

Factors which may be used in step (b), either singly or in combinations, are thus: pituitary extracts (e.g. a bovine pituitary extract), steroid hormones (e.g. hydrocortisone, or a salt thereof such as the acetate), growth factors (e.g. epidermal growth factor, preferably human form), catecholamines (e.g. epinephrine, either in racemic or enantiomeric form), iron-binding proteins (e.g. a transferrin), insulin, vitamins (e.g. retinoic acid), thyroid hormones (e.g. triiodothyronine), serum albumins (e.g. bovine or human serum albumin, including recombinant preparations), antibiotics (e.g. aminoglycoside antibiotics, such as gentamicin), and/or antifungals (e.g. amphotericin-B). Optimum sub-combinations of these components can easily be determined by culturing EBs in their presence and assessing the effect on differentiation towards a SPC^{+ve} phenotype, as described in the examples below. Other growth factors which may be used include: fibroblast growth factor 1 (FGF 1); FGF 7 (also known as keratocyte growth factor); FGF 10; and hepatocyte growth factor.

Preferred media for use in step (b) include epidermal growth factor. The medium may lack retinoic acid, pituitary extract and/or epinephrine.

It is surprising that SPC^{+ve} cells develop in the presence of hydrocortisone as this hormone has previously been reported to suppress SPC gene expression in AE2 cells [23].

As in step (a), cells are preferably grown in serum-free medium in step (b) and, if serum is not used, a serum replacement may be used instead.

Taking time zero as the time at which EBs are exposed to appropriate differentiation factors, step (b) will generally last between 1 and 30 days, and typically at least 3 days (e.g. around 7-14 days).

Cells will usually grow in step (b) in adherent culture. It is preferred to maintain adherent culture for all or substantially all of step (b). A typical starting concentration for step (b) is between 10⁴ and 10⁵ cells per ml of medium.

The process of the invention produces SPC^{+ve} cells from stem cells, via EBs. The cells are described in more detail below. To maintain the SPC^{+ve} cells after differentiation from EBs, serum-containing or serum-free media may be used.

Overall, the cells obtained from step (b) may be a heterogeneous population e.g. containing SPC^{+ve} cells of the invention, but also containing SPC^{-ve} cells. If a more homogenous population is desired, the product of step (b) can be further treated to remove contaminating cells. Cell enrichment and selection methods are familiar to the skilled person and include the use of flow cytometry (e.g. based on AE2-specific markers), of genetic selection, of selective growth media, or of selective killing. In this way, >95% pure cells may be obtained.

The efficiency of the process of the invention can be measured by comparing the number of SPC^{+ve} cells (output) with the number of stem cells (input). The process is preferably at least 10% efficient (i.e. at least 10% of the stem cells differentiate into SPC^{+ve} cells) or higher (e.g. 20%, 30%, 40%, 50%, 60%, 70%, 80%, 90%, 95% or more). Thus the invention provides a process for differentiating stem cells into SPC^{+ve} cells, wherein the proportion of stem cells which become SPC^{+ve} cells is ≧10% (e.g. 20%, 30%, 40%, 50%, 60%, 70%, 80%, 90%, 95% or more).

The Starting Stem Cells

The stem cell used in the process of the invention can be any pluripotent or multipotent stem cell. Pluripotent cells have the ability to develop into any cell derived from the three main germ cell layers. Adult stem cells, placental stem cells, fetal stem cells and umbilical stem cells may all be used, but preferred stem cells are embryonic stem (ES) cells, embryonic carcinoma (EC) cells or embryonic germ (EG) cells [e.g. 24, 25]. Somatic, bone marrow and cord blood stem cells may be used, particularly where autologous AE2 cells are desired.

Methods for obtaining suitable stem cells and for maintaining them (e.g. in an undifferentiated state) prior to use in the process of the invention are well known.

ES cells are cells derived from embryos which can propagate indefinitely in in vitro culture. ES cells are pluripotent i.e. they have the ability to give rise in vivo to all cell types which comprise the adult animal. Murine ES cells [e.g. ref. 26] and human ES cells [e.g. refs. 27 & 28] are readily available and conditions for their undifferentiated growth are well known [e.g. refs. 29 to 39]. ES cells are properly referred to as pluripotent rather than totipotent, as they are incapable of forming some non-embryonic cell types.

In order to ensure compatibility with human patients, human stem cells, and human ES cells in particular, are preferred for use according to the invention. Although it has not yet reached the levels of murine ES cells, knowledge on the growth and differentiation of human ES cells is advanced [e.g. refs. 16 & 40-42]. Where non-human patients are to be treated or studied, however, stem cells from other organisms (e.g. from non-human primates or from mice) may be used. Non-human stem cells may also be used with humans in conjunction with xenotransplantation compatibility techniques.

For administration to humans, it may be preferred to use autologous ES cells. These may be prepared by, for instance, preparing an embryo by somatic cell nuclear transfer from a patient, and deriving ES cells from the embryo. Autologous somatic stems cells may also be used.

Because the provision of large quantities of material for therapeutic use is advantageous, the stem cell is preferably capable of prolonged proliferation in vitro.

The stem cell is preferably a human ES cell line which is eligible for US federal funding according to criteria outlined by President Bush in his address of 9th August 2001. More preferably, the stem cell is one which can be obtained from the NIH Human Embryonic Stem Cell Registry [43].

Artificial Alveolar Tissue

The process of the invention produces SPC^{+ve} cells from stem cells, via EBs. Thus the invention provides an isolated SPC^{+ve} cell differentiated in vitro from a stem cell, such as an ES cell.

SPC expression is a characteristic of AE2 cells, but the cells produced according to the invention do not necessarily show all the characteristics of AE2 cells. As well as being SPC^{+ve}, it is preferred that the cells produced by the method of the invention have one or more of the following phenotypes: SPA^{+ve}; SPB^{+ve}; SPD^{+ve}; possesses lamellar bodies in the cytoplasm; capable of synthesising and secreting (and preferably recycling) pulmonary surfactant; able to differentiate into AE1 cells; phagocytic; able to proliferate; can bind to AE1 cells; can bind to fibroblasts; cuboidal shape; capable of secreting lysozyme; expresses one or more of alkaline phosphatase, catalase, Na⁺/K⁺ATPase, cytochrome P450 monooxygenase, glycoproteins recognised by Maclura pomiflerae lectin; stains positive with modified Papanicolaou method, phosphine 3R, tannic acid; and/or capable of regulating hypophase pH and [Ca²⁺]. The cells are most preferably AE2 cells.

These phenotypic properties may be manifested in vitro and/or in vivo, and it is preferred that they are manifested in vivo.

The cells of the invention are preferably murine or human cells.

The cells of the invention are preferably non-tumorigenic. They may have a normal karyotype.

The cells of the invention may be clonal.

Cells of the invention may be in the form of an aggregate or cluster, or in the form of separate cells. Cells in a cluster are preferably present in three dimensions (i.e. not as a single layer of cells).

The invention provides a collection of cells, wherein the collection comprises cells of the invention. Within the collection, at least 95% of the total number of cells (e.g. at least 96%, 97%, 98%, 99%, 99.5%, 99.9% or more) should be SPC^{+ve} cells of the invention (e.g. 100%). Thus the invention provides a collection of cells which are entirely SPC^{+ve} cells. Previous methods of purifying AE2 cells from lung tissue [e.g. refs. 6 to 10] do not achieve more than about 95% purity.

The cells in the collection are preferably of a single genotype or haplotype.

The collection or cluster preferably lacks undifferentiated starting stem cells.

The collection or cluster preferably lacks epithelial cell types, fibroblasts and/or endothelial cells (e.g. vasculature).

The collection preferably includes at least 1000 SPC^{+ve} cells.

The invention provides a collection of cells, wherein the collection comprises SPC+ve cells, and wherein the number of said SPC^{+ve} cells per gram weight of all cells in the collection is at least 10⁸ (e.g. at least 10⁹, at least 10¹⁰, at least 10¹¹, at least 10¹² etc.).

A collection of cells may additionally include AE1 cells and/or mesenchyme. A preferred collection of cells comprises AE2 cells, AE1 cells, vasculature and supporting matrices, arranged substantially identically to natural lung tissue.

The cells, collections and clusters of the invention may be located in vitro or in vivo. When in vitro, they may be in isolated form.

The cells, and collections and clusters thereof, are useful for pulmonary therapy and for in vitro studies of alveolar function.

The invention provides lung tissue comprising a cell of the invention. In particular, the invention provides comprising lung tissue comprising a cell of the invention at an alveolar surface thereof.

The invention provides a cell culture comprising SPC^{+ve} cells, wherein the mass of said cells within the culture is at least 10 grams (e.g. 20, 50, 100, 500, 1000, 2000, 10000 g).

The invention provides a cell culture comprising SPC^{+ve} cells, wherein the proportion of said SPC^{+ve} cells compared to the total number of cells in the medium is 95% or greater (e.g. 96%, 97%, 98%, 99%, 99.5%, 99.9% or more).

The invention provides an isolated SPC^{+ve} cell, wherein the telomeres in the cell are longer than the telomeres in a SPC^{+ve} cell found in vivo in lung tissue of the same species.

Therapeutic Methods

Cells of the invention are suitable for inducing lung tissue regeneration in vivo. Thus the invention provides a method of treating a patient, comprising administering cells of the invention to the patient.

The invention also provides the cells of the invention for use as a medicament.

The invention also provides the use of the cells of the invention in the manufacture of a medicament for treating a patient.

For therapy, the cells are preferably in the form of single cells, rather than a cluster or a collection of cells of the invention.

The invention also provides a syringe containing cells of the invention.

Cells of the invention may be encapsulated e.g. in an alginate hydrogel [44] or in any other biodegradable and biocompatible material (e.g. poly(lactide-co-glycolide) ‘PLG’). The encapsulated cells may include appropriate soluble factors e.g. for promoting engraftment in the lung, or for repressing differentiation of AE2 cells to AE I cells (see below) prior to engraftment.

Cells are preferably administered to the distal lung of a patient. They may be administered direct to the distal lung, or they may be delivered remotely, after which the cells are transported to the lung where they ‘dock’.

Cells of the invention are also suitable for inducing lung tissue regeneration ex vivo, for instance in tissue prior to transplant. Thus the invention provides an ex vivo method for modifying lung tissue, comprising adding a cell of the invention to said lung, preferably to an alveolar surface.

The number of cells to be delivered in vivo or ex vivo is based on a number of parameters, including: the body weight of the recipient, the severity of disease, and the number of cells surviving within the subject. A typical number of cells may be around 10⁷ to 10⁸ cells per kg body weight. It may be necessary to repeat infusions of the cells over several months to achieve the necessary cumulative total mass and/or to replace cells which are dying.

The cells will be delivered in combination with a pharmaceutical carrier. This carrier may comprise a cell culture medium which supports their viability. The medium will generally be serum-free in order to avoid provoking an immune response in the recipient. The carrier will generally be buffered and/or pyrogen-free.

Procedures for the preparation of cells of the invention for transplantation into a subject may include methods of protecting cells against an inflammatory response by the transplant recipient. For instance, the cells of the invention may be co-administered with anti-inflammatory agent(s). The cells may be delivered in conjunction with an anticoagulant (e.g. heparin) and/or with human serum albumin (preferably recombinant), typically in the same injection. Encapsulation may also be used, as described above.

The cells of the invention are preferably matched (e.g. by HLA-typing) to the recipient patient in order to avoid rejection. This can conveniently be achieved by preparing the cells from stem cells which are compatible with the recipient. For example, the stem cells could be derived from the recipient e.g. adult stem cells, or autologous ES cells prepared as described above. If cells are not well matched, they may be administered together with an appropriate immunosuppressant.

Included within the invention are methods and uses in which immature cells are transplanted into a recipient, where they subsequently mature into cells of the invention. Unlike the mature cells, the immature cells may not themselves be SPC^{+ve} cells, although they will become so in vivo.

In vitro Investigative Methods

The cells of the invention are essentially artificial alveolar tissue, although they do not necessarily exhibit all the characteristics of alveoli. Because of their ability to secrete SPC (and, indeed, other components of pulmonary surfactant), the cells can be used instead of alveolar tissue in in vitro screening assays. Cells of the invention are thus suitable for in vitro studies of alveolar tissue.

The invention provides an in vitro assay (e.g. for assessing the effect(s) of a test substance on alveolar pneumocytes), comprising the steps of (a) incubating a test substance with cells of the invention, and (b) detecting changes in said cells.

The substance may be a single compound or a mixture of compounds. Typically, it will be a pollutant (e.g. occupational or environmental) or a toxin. Components of cigarette smoke may be used.

Step (b) involves comparing cells after treatment with the substance to starting cells, with changes being caused by exposure to the substance. Various methods may be used for detecting the changes. At one extreme, detection of cell death (e.g. by necrosis, lysis, apoptosis etc.) is easily detected. Where cell death does not occur, other changes may be detected, such as: changes in morphology e.g. detected by microscope; changes in gene expression e.g. detected by Northern blotting, by SAGE, by microarray profiling etc.; changes in changes in protein expression e.g. detected by Western blotting, by immunoassay, by 2D-PAGE etc.; changes in genetic structure e.g. detected by standard genotoxicity assays; etc. The change may be one or more of the following: secretion of pulmonary surfactant; up-regulation of SPA, SPB, SPC and/or SPD transcription and/or translation; or cell proliferation and/or differentiation. The change may be monitored directly or indirectly.

The changes in step (b) may also be used to provide information on the etiology of pulmonary disease in the alveolar region of the lung.

Cells of the invention may also be used for investigating mechanisms of lung repair and/or regeneration. For instance, known pulmonary toxins could be administered to cells of the invention and then test substances could be assayed for their ability to ameliorate the toxicity.

Cells are also useful for screening for pharmacologically active substances. In such cases typical test substances are compounds which include, but are not restricted to, peptides, peptoids, proteins, lipids, metals, nucleotides, nucleosides, small organic molecules, antibiotics, polyamines, and combinations and derivatives thereof. Small organic molecules have a molecular weight of more than 50 and less than about 2,500 Da, and most preferably between about 300 and about 800 Da. Complex mixtures of substances, such as extracts containing natural products, or the products of mixed combinatorial syntheses, can also be tested and the component that causes secretion can be purified from the mixture in a subsequent step.

Test substances may be derived from large libraries of synthetic or natural compounds. For instance, synthetic compound libraries are commercially available from Maybridge Chemical Co. or Aldrich. Alternatively, libraries of natural compounds in the form of bacterial, fungal, plant and animal extracts may be used. Additionally, test compounds may be synthetically produced using combinatorial chemistry either as individual compounds or as mixtures.

The screening method may be in a high-throughput format. Preferably, all the biochemical steps for this assay are performed in a single solution in, for instance, a test tube or microtitre plate, and the test compounds are analysed initially at a single compound concentration. for the purposes of high throughput screening, the experimental conditions are adjusted to achieve a proportion. of test compounds identified as “positive” compounds from amongst the total compounds screened. The assay is preferably set to identify compounds with an appreciable affinity towards the target e.g., when 0.1% to 1% of the total test compounds from a large compound library are shown to bind to a given target with a Kᵢ of 10 μM or less (e.g. 1 μM, 100 nM, 10 nM, or less)

The invention also provides a compound identified by the method of the invention, optionally in combination with a pharmaceutical carrier. The compounds may themselves be useful pharmaceuticals or they may be useful lead compounds for development into pharmaceuticals. They are preferably organic compounds. The invention provides a compound identified by the method of the invention for use as a medicament, and the use of a compound identified by the method of the invention in the manufacture of a medicament for treating alveolar dysfunction.

AE1 Cells

AE2 cells can proliferate and can differentiate into AE1 cells [5]. Thus the invention provides a method for preparing an AE1 cell, comprising the step of culturing a cell of the invention under appropriate conditions. The process preferable occurs in vitro.

Genetic Manipulation of Cells

A stem cell may have been genetically manipulated prior to use in the process of the invention. Similarly, differentiated derivatives of the stem cells may be genetically manipulated after the process of the invention has been performed. This may involve activation or over-expression of endogenous genes and/or introduction of exogenous genes.

Suicide genes A cell may have been genetically manipulated to include a “suicide gene”. This provides a method of selectively killing cells which may persist in cell preparations to be administered to patients (e.g. undifferentiated stem cells), or all cells (differentiated or undifferentiated) derived from the stem cells as a failsafe mechanism to destroy the cells after transplantation. Suicide genes encode protein products that have no appreciable direct effect on cellular function, but which are capable of conferring toxicity by their ability to convert otherwise non-toxic substances (frequently termed prodrugs) into toxic metabolites. Suicide gene technology has been developed as a means of rendering cancer cells more sensitive to chemotherapeutics and also as a safety feature of retroviral gene therapy. Several combinations of suicide genes and prodrugs are known in the art [e.g. ref. 45] and include: E.coli cytosine deaminase+5-fluorocytosine; HSV thymidine kinase+ganciclovir or acyclovir; E.coli nitroreductase+CB1954 etc. The suicide gene is preferably under the control of a promoter expressed in undifferentiated stem cells or in other cells undesirable for transplantation (e.g. tumors or tumorigenic cells), in which case undifferentiated cells can be removed from culture by using the appropriate prodrug without affecting differentiated cells. For use as a failsafe mechanism to allow selective killing of a transplant in a patient (e.g. where the transplant is found to be harmful in a recipient), however, the suicide gene will generally be under the control of a constitutive promoter, although tissue-specific or inducible promoters may be used.

Differentiation promotion A stem cell may have been genetically manipulated to encode a polypeptide (e.g. a transcription factor) which promotes differentiation of the stem cell into a SPC^{+ve} cell in step (a) or step (b). The same strategy may be used to maintain and/or stabilise a SPC^{+ve} phenotype. Expression of this polypeptide may be controlled so that it occurs in the stem cell itself, or so that it occurs in a derivative of the stem cell (e.g. in EBs). Conversely, a cell may have been genetically manipulated such that it under-expresses or does not express a polypeptide (e.g. a transcription factor) which either favours differentiation of the stem cell away from a SPC^{+ve} phenotype or which inhibits differentiation into a SPC^{+ve} cell. For instance, genes could be knocked out, or could be inhibited using antisense or RNA silencing (e.g. RNAi [e.g. refs. 46-49]) techniques.

Lineage selection and enrichment A cell may have been genetically manipulated to insert markers suitable for lineage selection, a technique which specifically selects a desired cell type e.g. based on a previously-inserted recombinant construct which comprises a tissue-specific promoter linked to a selectable marker. Where the efficiency of differentiation of stem cells into cells of the invention is inherently low, this technique allows efficiency to be increased. Suitable selectable marker genes include, but are not restricted to, drug selectable genes (e.g. the G418 resistance gene, zeo, bsd, HPRT, hygro, puro), visible markers such as fluorescent proteins (e.g. GFP, DsRed) and genes which facilitate selection by automated cell sorting (e.g. genes encoding cell surface antigens).

A cell may have been genetically manipulated to insert markers suitable for enrichment of cells from within a heterogeneous population. One possibility is to place a marker protein under the control of an AE2-specific promoter (e.g. the SPC promoter) such that the marker is transcribed in parallel to natural AE2-specific genes. The marker can then be detected and used for enriching cells of the invention (e.g. by FACS).

Compatibility A cell may have been genetically manipulated so that expression of reactive antigens is reduced or eliminated. For instance, genes which encode auto-antigens could be knocked out, or they could be inhibited using antisense techniques or RNA silencing (e.g. RNAi).

The genetic manipulations described above may be used singly, or two or more may be used in combination.

Genetic manipulation of the cell may occur through random integration into the genome or by gene targeting. As an alternative the manipulation may, where appropriate, use an episomally-maintained vector (e.g. a plasmid). Transfection of ES cells, including human ES cells [21], is well known.

For random integration, vector(s) which encode the relevant polypeptides may be introduced into the cell. Typically, an expression vector comprising a gene promoter operably linked to DNA encoding the relevant polypeptide would be used. The coding DNA may be cDNA, genomic sequences or a mixture of both. The promoter may direct constitutive or inducible expression and may be tissue-specific. Examples of constitutive promoters include the promoters from β-actin, phosphoglycerate kinase (PGK), glycolytic enzymes, elongation factor 1α (EF1α), or SV40. Examples of inducible gene promoters include systems composed of a chimeric transactivator that reversibly binds to the promoter region of the expression construct in response to a drug or ligand (e.g. doxycycline, ecdysone, mifepristone, tetracycline, FK1012, or rapamycin).

An alternative to random integration is the precise alteration of genes in situ by homologous recombination, termed “gene targeting”. This is the precise predetermined modification of genes by homologous recombination between exogenous and endogenous DNA. Gene targeting can be used to insert, replace, rearrange or remove chosen DNA sequences in cultured cells, most commonly embryonic stem cells [e.g. ref. 50]. In some circumstances gene targeting may be preferable to simple introduction of an expression vector at a random site because the genetic modification can be predetermined to avoid any deleterious effect (e.g. oncogenic transformation) that would reduce the therapeutic value of derived cells.

Gene targeting may be used to achieve constitutive or inducible expression of a gene of interest by modifying or replacing the natural promoter or other regulatory regions of that gene. For example, a gene promoter may by replaced with a constitutive or inducible promoter or elements which direct constitutive expression may added adjacent to the endogenous gene promoter. Methods to achieve such modifications by gene targeting, including in ES cells, are well known in the art.

It is also possible to perform genetic manipulation on a cell other than a stem cell, and then to transfer that genetic manipulation into a stem cell (e.g. by transfer of a nucleus into an enucleated stem cell) or into an embryo (e.g. by transfer of a nucleus into an enucleated oocyte) which can give rise to a stem cell. Both of these approaches indirectly give a genetically-manipulated stem cell.

Alternative Step (a)

As an alternative to forming EBs in step (a), the stem cells may be co-cultured with lung mesenchyme. Embryonic lung mesenchyme is particularly suitable.

### BRIEF DESCRIPTION OF THE DRAWINGS

FIGS. 1 and 2 show phase contrast microscopy of differentiating ES cells at confluence after trypsinization and subculture in (1) ES cell medium or (2) SAGM medium. The bar is 500 μm.

FIG. 3 shows PCR amplification of β-actin gene in a single sample with increasing cycle numbers.

FIGS. 4 to 6 show RT-PCR results for cells differentiated through EBs. FIG. 4 shows the results of a first PCR amplification, with FIG. 5 showing the results of a second nested amplification. FIG. 6 shows the presence of β-actin.

FIG. 7 shows the results of Stul digestion of the PCR products from FIGS. 4 to 5.

FIGS. 8 to 10 show the results of RT-PCR for cells differentiated without EB formation. The figures parallel FIGS. 4 to 6.

FIG. 11 is a high power photomicrograph showing an ES cell-derived cell cluster grown in SAGM, immunostained for SPC (bar=80 μm).

FIG. 12 is an electron micrograph of a cytoplasmic organelle within such a cluster, with the characteristic structure of a AE2 lamellar body visible (bar=100 nm).

### MODES FOR CARRYING OUT THE INVENTION

Culture and Differentiation of ES Cells

Murine E14Tg2a ES cells [51; Dr A. Smith, University of Edinburgh, Scotland] were grown in an undifferentiated state on gelatin-coated tissue culture plates in the presence of 1000 U/ml LIF. Cells were maintained in ‘ES cell medium’ [Glasgow Minimum Essential Medium (G-MEM; available as catalogue number 11710 or 22100 from Invitrogen Life Technologies) supplemented with 10% fetal calf serum (FCS), 2 mM L-glutamine, 0.1 mM 2-mercaptoethanol, and antibiotics (penicillin 100 U/ml; streptomycin 100 μg/ml); no tryptose broth].

To promote AE2 cell formation, ES cells were first induced to differentiate spontaneously in ES cell medium via EB formation. For this, confluent cultures of undifferentiated ES cells were subjected to limited trypsin digestion (0.05% trypsin; 0.53 mM EDTA in 0.1 M PBS without calcium or magnesium; 2% chicken serum) to produce clusters of 8-15 cells, which were cultured in non-adherent bacterial grade Petri dishes in ES cell medium without LIF (day 0). Most EBs adhered to the dish surface after 8-10 days of culture and the cells spread out. The differentiating ES cells were fed on alternate days with ES cell medium for periods of 10-, 20- or 30-days. At the end of each time period, cells were either maintained in ES cell medium or were transferred into ‘SAGM’ medium (Biowhittaker, Watersville, Md.) designed for the growth and maintenance of mature distal lung epithelium, at an initial cell seeding density of 5×10⁴ cells/cm². SAGM consists of basal medium plus the following factors: 0.5 mg/ml bovine serum albumin, 5 μg/ml insulin, 10 μg/ml transferrin, 30 μg/ml bovine pituitary extract, 0.5 μg/ml epinephrine, 6.5 ng/ml triiodothyronin, 0.1 ng/ml retinoic acid, 0.5 μg/ml hydrocortisone, 0.5 ng/ml human epidermal growth factor and antibiotics (gentamycin sulphate 0.05 mg/ml; amphotericin-B 0.05mg/ml). Following incubation for 3 or 14 days, as arbitrary short and long growth intervals, cells were collected for analyses.

In parallel experiments, ES cells were induced to differentiate by LIF removal alone without formation of EBs. This process encourages accumulation of primitive endoderm-like cells on the outside of undisrupted ES cell colonies. Cells were passaged continually several times (from passage number 13 through to 20) before being maintained in ES cell medium or transferred to SAGM and sampled as for EB-derived cultures.

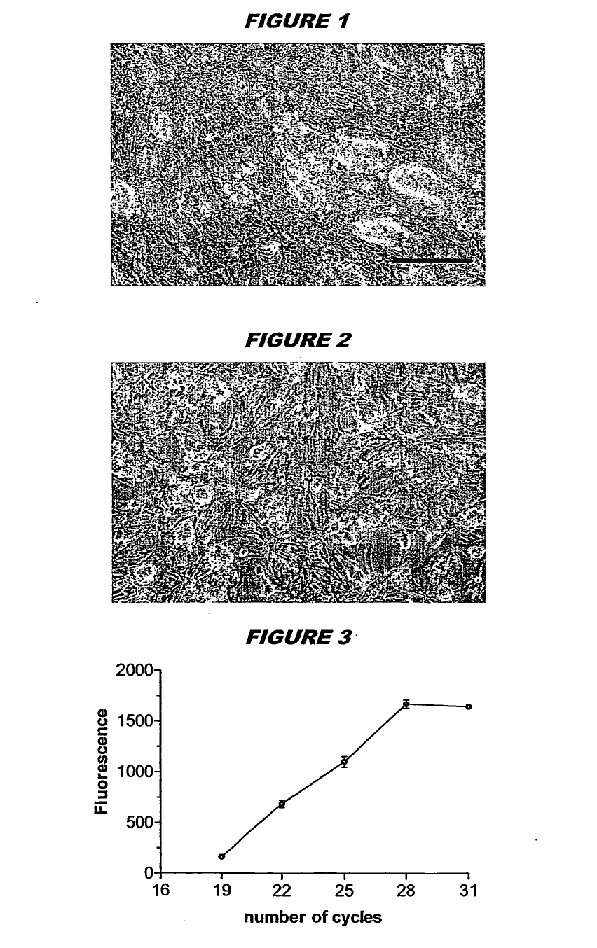

Undifferentiated ES cells grew in tightly packed colonies. Routine daily examination revealed changes in the appearance of the cultures following induction of spontaneous differentiation, regardless of the route used i.e. whether via EB formation or LIF removal alone. In outgrowths of cells from each adherent EB, it was possible to see differentiation into a variety of cell types, including epithelial cells and twitching muscle cells. However, following trypsinization and subsequent culture in the same medium (ES cell medium), the cells appeared to be organized into two areas with distinct morphologies—small round cells with a large nucleus-to-cytoplasm ratio arranged in groups or islands, and others with fibroblastic appearance filling the spaces between groups of small cells (FIG. 1). Transfer into SAGM caused a reduction in the number of small groups of cells, presumably due to cell death, and resulted in the spaces between the groups being filled with fewer and more flattened cells (FIG. 2).

During culture, cells were examined daily under an inverted light microscope. At the end of each period of culture in the two different media, cells were sampled in order to detect type II pneumocytes by:
a) reverse transcriptase-polymerase chain reaction (RT-PCR) for SPC mRNA, a specific marker for this cell type,
b) immunocytochemistry for SPC and
c) transmission electron microscopy (TEM) for detection of lamellar bodies, which are the sites of surfactant protein storage and are thus characteristic of AE2 cells.
   SPC mRNA Expression

RT-PCR was carried out on RNA extracted from differentiating ES cells cultured in ES cell medium or SAGM after 10-, 20- and 30-days of differentiation and growth in ES cell medium. Total RNA was extracted from the cultured cells using TRIzol reagent (Life Technologies) following the manufacturer's instructions. Thermoscript RT-PCR System (Life Technologies) was used to synthesize cDNA from 1 μg total RNA. Oligo(dT)₂₀ was used to prime RT reactions which enabled the same cDNA to be PCR amplified with different sets of gene-specific primers. 1 μl cDNA samples (5% of RT reaction) were amplified with primers selective for murine SPC and β-actin using the hot start enzyme Gold DNA polymerase (PE Applied Biosystems Inc). To ensure that only cDNA and not any potential contaminating genomic DNA was amplified, SPC PCR primers were designed to separate exons or to anneal across exon/exon boundaries. Primer sequences and the amplicon length are given in the following table:

| Amplified | | | SEQ | Amplicon |
|---|---|---|---|---|
| sequence | Name | Description | ID | length (bp) |
| SPC | SPC1 | Forward primer on exon 3/4 | 1 | 295^{a} |
| | SPC2 | Forward primer on exon 4 | 2 | 252^{a} |
| | SPC3 | Reverse primer on exons 6/5 | 3 | — |
| β-actin | β1 | Forward primer | 4 | 495^{b} |
| | β2 | Reverse primer | 5 | — |

^{a}length when used in conjunction with SPC3
^{b}length when used in conjunction with β2

In the 30 μl PCR reaction mix, the final concentrations of MgCl₂ and dNTPs were 2.5 mM and 0.1 mM, respectively. DNA amplification was performed in a Perkin Elmer GeneAmp PCR System 2400. Double-stranded DNA denaturation and the activation of AmpliTaq Gold DNA Polymerase was carried out at 94° C. for 10 minutes, followed by 35 cycles of: template denaturation at 94° C. (5 s), primer annealing at 56° C. (10 s) and primer extension at 72° C. (30 s). 1 μl of PCR reaction mix was amplified for further 15 cycles in a nested PCR reaction. A period of 7 minutes post-PCR extension at 72° C. was carried out for both PCR reactions. PCR products were separated on 2% agarose gel and visualised by ethidium bromide fluorescence.

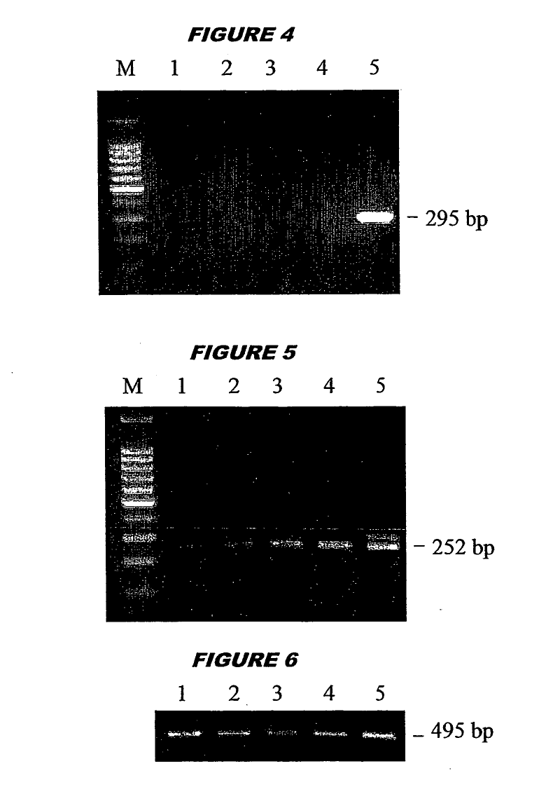

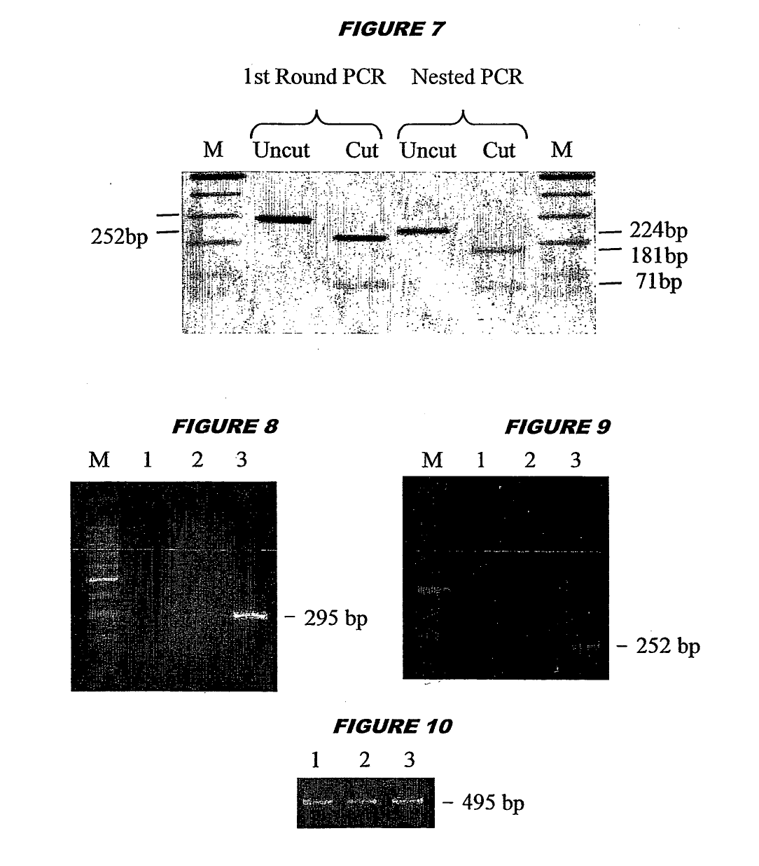

FIG. 4 shows the SPC amplicons after 35 cycles of PCR from cDNA synthesized from the equivalent of 50 ng of total RNA in cells initially cultured in ES cell medium for 30 days. Cells cultured in ES cell medium are shown in lanes 1 and 2 (3 and 14 days culture in ES cell medium, respectively). Cells cultured in SAGM are shown in lanes 3 and 4 (3 and 14 days culture). Lane 5 is a positive control amplified from adult murine lung cDNA. Weak bands are visible in lanes 3 and 4, but no bands were visible in cells grown only in ES cell medium. Therefore only SAGM-treated cells expressed SPC.

SPC expression was further studied by nested PCR for an additional 15 cycles. Results are shown in FIG. 5, with the lanes being the same as in FIG. 4. SPC amplicons are visible in cells grown solely in ES cell medium, but at a much lower level than for cells cultured in SAGM (lanes 3 and 4).

The nested PCR results confirmed the specificity of the SPC amplicon, and specificity was further verified by analysis of Stul restriction enzyme digests. As expected, Stul cut once to give products of 224 bp+71 bp and 181 bp+71 bp for the SPC and nested amplicons, respectively (FIG. 5).

To determine non-saturating PCR conditions for β-actin, the gene was amplified for 19, 22, 25, 28 & 31 cycles in triplicate using RT-material from a single sample first. Digital images of ethidium bromide-stained gels were captured using the Bio-Rad Fluor-S MultiImager system, which consists of an enclosed flat-bed UV light scanner and CCD camera, connected to a computer. Images were analyzed using Bio-Rad Quantity One quantitation software, which allows detection of the individual bands and subtraction of background noise, yielding intensity values due solely to the gene-specific amplified products. The linear range of PCR amplification was best after 22 cycles (FIG. 3). PCR was thus performed for 22 cycles on all cDNA samples, and revealed similar levels of the housekeeping gene, β-actin, for all four treatments (FIG. 6). The mean sample fluorescent intensities were 486±27, 448±30, 462±19, 483±78 (SEM; N=3) (FIG. 6, lanes 1-4).

For cells initially cultured in ES cell medium for 10 or 20 days and then transferred to SAGM, bands could not be detected either with 35 cycles of amplification or with nested PCR.

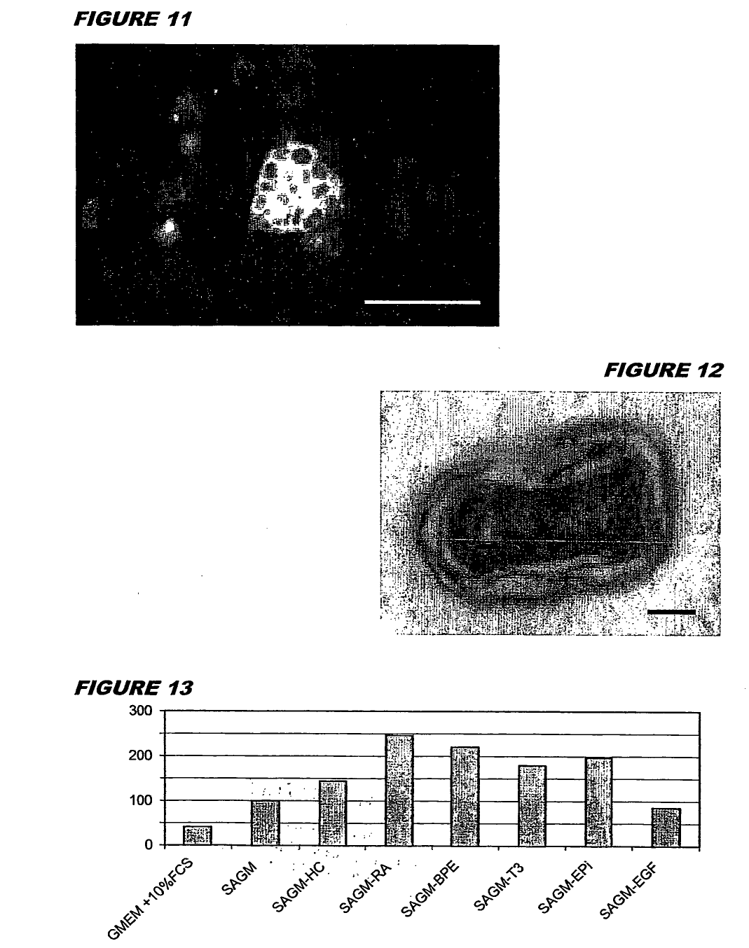

For cells which did not progress through EBs, SPC amplicons were not detected. FIG. 9 shows the lack of amplicon after 35 cycles of PCR, and FIG. 10 shows the same lack after additional nested PCR. Lane 1 shows cells cultured in ES cell medium after LIF removal and lane 2 shows cells cultured in SAGM after LIF removal. Lane 3 is a positive control (adult murine lung cDNA). FIG. 11 shows that β-actin cDNA was present in all samples.

Control reactions in which reverse transcriptase was omitted from the RT step were negative. Therefore optimum conditions for producing SPC^{+ve} cells involve: (a) differentiation via EBs; (b) more than 20 days of culture, and preferably at least 30 days, for producing the EBs; (b) transfer of the EBs into SAGM medium followed by culture for more than 3 days.

SPC Protein Expression

Cells grown in multi-chamber slides were rinsed twice in phosphate-buffered saline and fixed in 4% paraformaldehyde at room temperature for 30 minutes. Immunofluorescence staining of cultured cells was carried out using rabbit polyclonal antibodies to SPC (Chemicon, Temecula, Calif.; overnight incubation at dilution 1:200). The second layer comprised FITC-conjugated goat anti-rabbit IgG (dilution 1:200; Sigma). Preparations were viewed under an Olympus BX-60 microscope.

Scattered immunoreactive cells, single or in clusters, were seen in cultures grown in either ES cell medium or SAGM. FIG. 11 shows a cell grown in SAGM (bar=80 μm).

Variability in the quality of the immunostaining and the clumping of cells precluded any quantitative analysis.

Cellular Ultrastructure

Further confirmation of alveolar type epithelial differentiation came from cellular ultrastructure studies. Cells were rinsed twice in phosphate-buffered saline and fixed in 2.5% glutaraldehyde in 0.1M phosphate buffer pH 7.25 for 25 minutes, washed three times in 0.1 M phosphate buffer with 0.1M sucrose and post fixed in 1% osmium tetroxide in 0.1 M phosphate buffer. Following two washes in the phosphate buffer with sucrose, the cells were scraped from the wells, transferred to microfuge tubes and centrifuged. Pellets were dehydrated through a graded series of alcohols, followed by propylene oxide and embedded in Araldite resin. Ultra-thin sections of silver-gold interference colour were stained with 4% uranyl acetate in methanol and observed in a Zeiss 10 CR electron microscope.

FIG. 12 (bar=100 nm) shows that cells include osmiophilic lamellar bodies composed of AE2-typical concentric laminae after growth in SAGM. In addition, extrusion of tubular myelin could sometimes be seen.

SAGM Dissection

SAGM consists of a serum-free basal medium supplemented with: BPE, hydrocortisone (HC), HEGF, epinephrine (EPi), transferrin, insulin, retinoic acid (RA), triiodothyronine (T3), gentamicin, amphotericin-B and BSA-FAF. Six of these components were removed singly from SAGM and the effect on AE2 derivation was assessed by quantitative PCR of SPC mRNA (FIG. 13).

Compared with basic medium (GMEM+10% fetal calf serum), SAGM showed a >100% increase in SPC MRNA. Removal of most SAGM components increased SPC mRNA levels i.e. these factors have suppressive effects on SPC expression. Removal of HEGF, however, reduced SPC levels, suggesting that this growth factor stimulates SPC expression. SAGM minus the suppressive factors is a preferred medium.

In a later experiment, ES cells were grown in basic medium for 10 days and then kept either in the basic medium or transferred to (i) complete SAGM; (ii) SAGM minus HC; or (iii) SAGM minus RA. No SPC mRNA was detected in cells grown in basic medium or complete SAGM, but it was found in cells grown in modified SAGM, indicating differentiation to give AE2 cells.

It will be understood that the invention has been described above by way of example only and that modifications may be made whilst remaining within the scope and spirit of the invention.

References (the Contents of which are Hereby Incorporated in Full)

[1] Wright et al. (1984) pages 1068-1074 of The Biology of Epithelial Cell Populations (OUP).
[2] Solarin et al. (2001) Pediatr Pathol Mol Med 20:471-500.
[3] Johansson (1998) Biochim Biophys Acta 1408:161-172.
[4] Beers & Fisher (1992) Am J Physiol 263:L151-160.
[5] Fehrenbach (2001) Respir. Res. 2:33-46.
[6] Castranova et al. (1988) Toxicol Appl Pharmacol 93:472-483.
[7] Dobbs et al. (1986) Am Rev Respir Dis 134:141-145.
[8] Jassal et al. (1991) In Vitro Cell Dev Biol 27A:625-632.
[9] International patent application WO 01/42425.
[10] Corti et al. (1996) Am. J. Respir. Cell Mol. Biol. 14:309-315.
[11] Shannon et al. (1999) Development 126:1675ff.
[12] Desbaillets et al. (2000) Exp Physiol 85:645-51.
[13] Keller (1995) Curr Opin Cell Biol 7:862-9.
[14] Mueller-Klieser (1997) Am J Physiol 273:C1109-23.
[15] Magyar et al. (2001) Ann N Y Acad Sci 944:135-43.
[16] Zhang et al. (2001) Nature Biotechnol 19:1129-33.
[17] Schuldiner et al. (2000) PNAS USA 97:11307-11312.
[18] Carpenter et al. (2001) Exp Neurol 172(2):383-397.
[19] Schuldiner et al. (2001) Brain Res913(2):201-205.
[20] Kehat et al. (2001) J Clin Invest 108(3):407-414.
[21] Eiges et al. (2001) Curr Biol 11:514-518.
[22] See www.cambrex.com.
[23] Shannon et al. (2001) Am J Respir Cell Mol Biol 24:235-244.
[24] U.S. Pat. No. 6,090,622.
[25] Donovan & Gearhart (2001) Nature 414:92-97.
[26] U.S. Pat. No. 5,670,372.
[27] U.S. Pat. No. 6,200,806.
[28] Thomson et al (1998) Science 282, 1145-7.
[29] Smith (2001) Ann. Rev Cell Dev Biol 17:435-62.
[30] National Institutes of Health, Dept of Health and Human Services Report. Stem cells: scientific progress and future research directions. June 2001 (www.nih.gov/news/stemcell/scireport.htm).
[31] Robertson (1987) Teratocarcinomas and embryonic stem cells, a practical approach. IRL Press.
[32] Wobus (2001) Mol Aspects Med 22:149-64.
[33] Tessarollo (2001) Methods Mol Biol 158:47-63.
[34] Wobus et al. (2001) Methods Mol Biol 158:263-86.
[35] Marshall et al. (2001) Methods Mol Biol 158:11-18.
[36] Wobus et al. (2000) Cells Tissues Organs 166:1-5.
[37] Pera et al. (2000) J Cell Sci 113:5-10.
[38] Lebkowski et al. (2001) Cancer J 7 Suppl 2:S83-93.
[39] Embryonic Stem Cells: Methods and Protocols (ed. Turksen) 2002. ISBN 0896038815.
[40] Zhang et al. (2001) Nature Biotechnol. 19:1129-1133.
[41] Donovan et al. (2001) Nature 414:92-97.
[42] Pera (2001) Curr Opin Genet Dev. 11:595-599.
[43] http://escr.nih.gov/
[44] Siebers et al. (1997) Ann N Y Acad Sci 831:304-12.
[45] Springer & Niculescu-Duvaz (2000) J Clin Invest 105:1161-1167.
[46] Zamore (2001) Nat Struct Biol 8:746-750.
[47] Carthew (2001) Curr Opin Cell Biol 13:244-248.
[48] Billy et al. (2001) PNAS USA 98:14428-14433.
[49] Yang et al. (2001) Mol Cell Biol 21:7807
[50] Ledermann (2000) Exp Physiol 85:603-613.
[51] Hooper et al. (1987) Nature 326:292.

1. A process for causing a stem cell to differentiate into a cell which expresses surfactant protein C (SPC), the process comprising the steps of: (a) culturing the stem cell to give an embryoid body; and (b) culturing the embryoid body under conditions which cause it to differentiate into cells which express surfactant protein C.

2. The process of claim 1, wherein stem cells are grown initially in step in suspension culture.

3. The process of claim 1, wherein embryoid bodies formed in step (a) are exposed to differentiation factors.

4. The process of claim 1, wherein step (b) takes place in the presence of epidermal growth factor.

5. The process of claim 1, wherein step (b) takes place in the presence of SAGM.

6. The process of claim 1, wherein the stem cell is an embryonic stem (ES) cell.

7. A SPC^{+ve} cell differentiated in vitro from a stem cell.

8.-11. (canceled)

12. A method of treating a patient, comprising administering cells according to claim 7 to the patient.

13. Cells according to claim 7 for use as a medicament.

14. Use of cells according to claim 7 in the manufacture of a medicament for treating a patient.

15. A syringe containing cells according to claim 7.

16. Cells according to claim 7, wherein the cells are encapsulated in an artificial material.

17. (canceled)

18. An in vitro assay comprising the steps of (a) incubating a test substance with cells according to claim 7, and (b) detecting changes in said cells.

19. A method for preparing an AE1 cell, comprising the step of culturing cells according to claim 7 under appropriate conditions.

20. (canceled)

21. A process for causing a stem cell to differentiate into a cell which expresses surfactant protein C, the process comprising the steps of: (a) co-culturing the stem cell with lung mesenchyme; and (b) culturing the cell under conditions which cause it to differentiate into a cell which expresses surfactant protein C.


SEQUENCE LISTING (5 sequences)

SEQ ID NO: 1 - DNA, 20 bp, Artificial Sequence
  Primer
tatgactaccagcggctcct

SEQ ID NO: 2 - DNA, 26 bp, Artificial Sequence
  Primer
gaacctactgctacatcatgaagatg

SEQ ID NO: 3 - DNA, 20 bp, Artificial Sequence
  Primer
gtttctaccgaccctgtgga

SEQ ID NO: 4 - DNA, 24 bp, Artificial Sequence
  Primer
gtcgtaccacaggcattgtgatgg

SEQ ID NO: 5 - DNA, 22 bp, Artificial Sequence
  Primer
gcaatgcctgggtacatggtgg